(19) United States
(12) Patent Application Publication
(10) Pub. No.: US 2025/0205398 A1
(43) Pub. Date: Jun. 26, 2025

(54) MAGNETICALLY-REGENERATIVE STENT

(71) Applicant: BOSTON SCIENTIFIC SCIMED, INC., Maple Grove, MN (US)

(72) Inventor: Adam McGraw, Mansfield, MA (US)

(73) Assignee: BOSTON SCIENTIFIC SCIMED, INC., Maple Grove, MN (US)

(21) Appl. No.: 18/988,146

(22) Filed: Dec. 19, 2024

Related U.S. Application Data
(60) Provisional application No. 63/612,860, filed on Dec. 20, 2023.

(51) Int. Cl. A61L 31/12; A61F 2/04; A61L 31/04; A61L 31/06; A61L 31/14

(52) U.S. Cl. CPC A61L 31/128; A61F 2/04; A61L 31/044; A61L 31/06; A61L 31/145; A61F 2002/041; A61F 2210/0076; A61F 2210/009; A61L 2400/12

(57) ABSTRACT

An illustrative stent includes an elongated tubular member forming a tubular wall, the elongated tubular member configured to move between a radially collapsed configuration and a radially expanded configuration. A polymer coating may be disposed on a surface of the tubular wall. A thermo-responsive layer may be disposed on an inner surface of the polymer coating. Magnetic nanoparticles may be disposed on or within the thermo-responsive layer.

### CROSS REFERENCE TO RELATED APPLICATIONS

This application claims the benefit of and priority to U.S. Provisional Patent Application Ser. No. 63/612,860, filed on Dec. 20, 2023, the disclosure of which is incorporated herein by reference.

### TECHNICAL FIELD

The present disclosure pertains to medical devices, methods for manufacturing medical devices, and uses thereof. More particularly, the present disclosure pertains to a magnetically regenerative stent for implantation in a body lumen, and associated methods.

### BACKGROUND

Implantable stents are devices that are placed in a body lumen, such as the esophageal tract, the gastrointestinal tract (including the intestine, stomach and the colon), tracheobronchial tract, urinary tract, biliary tract, vascular system, etc. to provide support and to maintain the body lumen open. These stents are manufactured by any one of a variety of different manufacturing methods and may be used according to any one of a variety of methods. Of the known stents, delivery systems, and methods, each has certain advantages and disadvantages. For example, some stents may become occluded. Occlusive events in pancreatic and biliary environments can be a combination of local factors, such as, but not limited to inadequate stricture resolution and irregular bile properties that contribute to occlusive biofilm formation and/or related to stent design, such as, but not limited to, coating tackiness and/or an interaction between the wire or sheathing material and bile. Thus, there is an ongoing need to provide alternative stent designs which offer opportunities for prophylactic intervention to restore stent patency responsive to occlusive events or potential occlusive sources without surgical intervention, thereby increasing stent efficacy and/or an operational lifetime of the stent.

### BRIEF SUMMARY

This disclosure provides design, material, manufacturing method, and use alternatives for medical devices, such as a stent.

A first example is directed to a stent. The stent includes an elongated tubular member forming a tubular wall. The elongated tubular member is configured to move between a radially collapsed configuration and a radially expanded configuration. A polymer coating is disposed on a surface of the tubular wall. A thermo-responsive layer is disposed on an inner surface of the polymer coating. Magnetic nanoparticles are disposed on or within the thermo-responsive layer.

Alternatively or additionally to any of the examples herein, the polymer coating comprises silicone or polyurethane.

Alternatively or additionally to any of the examples herein, the polymer coating extends from a proximal end to a distal end of the elongated tubular member, and wherein the thermo-responsive layer extends from the proximal end to the distal end of the elongated tubular member.

Alternatively or additionally to any of the examples herein, the thermo-responsive layer comprises a thermo-responsive polymer or a thermo-responsive hydrogel.

Alternatively or additionally to any of the examples herein, the thermo-responsive layer comprises a collagen and polycaprolactone matrix.

Alternatively or additionally to any of the examples herein, the magnetic nanoparticles are spaced along a length of the thermo-responsive layer.

Alternatively or additionally to any of the examples herein, the magnetic nanoparticles are uniformly dispersed on or embedded within the thermo-responsive layer along a length of the thermo-responsive layer.

Alternatively or additionally to any of the examples herein, the magnetic nanoparticles are arranged with a higher concentration of magnetic nanoparticles along a first region of the elongated tubular member than a concentration of the magnetic nanoparticles along a second region of the elongated tubular member.

Alternatively or additionally to any of the examples herein, the first region is adjacent to one or more structures forming the tubular wall, and the second region is less proximate to the one or more structures forming the tubular wall.

Alternatively or additionally to any of the examples herein, the magnetic nanoparticles are dispersed on or are embedded within the thermo-responsive layer in a gradient extending from the first region to the second region.

Alternatively or additionally to any of the examples herein, the first region is located adjacent to a first end of the elongated tubular member and the second region is located adjacent to a second end of the elongated tubular member.

Alternatively or additionally to any of the examples herein, the magnetic nanoparticles have a number-average particle diameter in a range from about 1 nanometer to about 400 nanometers.

Alternatively or additionally to any of the examples herein, the magnetic nanoparticles comprise a silicone base and a magnetic material.

Alternatively or additionally to any of the examples herein, in response to an applied magnetic field, the magnetic nanoparticles are configured to be excited to an elevated temperature in a range from 42 degrees Celsius to 58 degrees Celsius, the thermo-responsive layer is configured with a critical solution temperature or a melting point in the range from 42 degrees Celsius to 58 degrees Celsius, and the applied magnetic field is a pulsed magnetic field or an alternating magnetic field.

Alternatively or additionally to any of the examples herein, the thermo-responsive layer comprises a plurality of thermo-responsive layers. The plurality of thermo-responsive layers include a second thermo-responsive layer disposed on an inner surface of the polymer coating and a first thermo-responsive layer disposed on an inner surface of the second thermo-responsive layer. The second thermo-responsive layer is configured to be excited to a second elevated temperature in response to an applied magnetic field. The first thermo-responsive layer is configured to be excited to a first elevated temperature in response to an applied magnetic field.

Another illustrative example is a stent. The stent includes an elongated tubular member forming a tubular wall. The elongated tubular member is configured to move between a radially collapsed configuration and a radially expanded configuration. A polymer coating is disposed on a surface of the tubular wall. A thermo-responsive layer is disposed on an inner surface of the polymer coating. Magnetic nanoparticles are disposed on or within the thermo-responsive layer. In response to one or more of the magnetic nanoparticles being excited to an elevated temperature by application of a magnetic field, at least a portion of the thermo-responsive layer is configured to liquify and mechanically decouple from a remaining portion of the thermo-responsive layer, the polymer coating, or both.

Another illustrative example is a method for restoring patency of a stent implanted in a body of a patient. The method includes applying a magnetic field to excite one or more magnetic nanoparticles in a thermo-responsive layer of the implanted stent to an elevated temperature, wherein the magnetic field is a pulsed magnetic field or an alternating magnetic field. In response to the one or more magnetic nanoparticles being excited to the elevated temperature, at least a portion of the thermo-responsive layer is liquified.

Alternatively or additionally to any of the examples herein, the liquified portion of the thermo-responsive layer is configured to mechanically decouple from a remaining portion of the thermo-responsive layer, a polymer coating of the implanted stent, or both.

Alternatively or additionally to any of the examples herein, the one or more magnetic nanoparticles are configured to be exited to an elevated temperature in a range from 42 degrees Celsius to 58 degrees Celsius, and the thermo-responsive layer is configured with a critical solution temperature or melting point in the range from 42 degrees Celsius to 58 degrees Celsius.

Alternatively or additionally to any of the examples herein, the portion of the thermo-responsive layer that is configured to be liquified is further configured to remain liquified subsequent to cessation of applying the magnetic field.

Alternatively or additionally to any of the examples herein, the magnetic field is applied exterior of the patient's body.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a side view of an illustrative endoluminal implant or stent;

FIGS. 2A-2C are side views of the illustrative endoluminal implant or stent having a thermo-responsive layer including a temperature-sensitive polymer or hydrogel layer with magnetic nanoparticles in different arrangements;

FIG. 3A is a schematic cross-sectional view of the endoluminal implant or illustrative stent having a thermo-responsive layer including a temperature-sensitive polymer or hydrogel layer with magnetic nanoparticles prior to magnetization, taken at line 1-1 of FIG. 1;

FIG. 3B is a schematic cross-sectional view of the illustrative endoluminal implant or stent during magnetization;

FIG. 3C is a schematic cross-sectional view of the illustrative endoluminal implant or stent after magnetization;

FIG. 4A is a schematic cross-sectional view of another illustrative endoluminal implant or stent having a plurality of thermo-responsive layers including magnetic nanoparticles prior to magnetization, taken at line 1-1 of FIG. 1;

FIG. 4B is a schematic cross-sectional view of the illustrative endoluminal implant or stent after magnetization at a first frequency;

FIG. 4C is a schematic cross-sectional view of the illustrative endoluminal implant or stent a period of time following the magnetization at the first frequency;

FIG. 4D is a schematic cross-sectional view of the illustrative endoluminal implant or stent after magnetization at a second frequency;

FIG. 5 is a schematic view of an illustrative endoluminal implant or stent deployed within the biliary tree; and

FIG. 6 is a schematic view of an illustrative placement of a console for generating a magnetic field relative to a body of a patient.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

In some instances, it may be desirable to provide an endoluminal implant, or stent, that can deliver luminal patency within the pancreaticobiliary tree of a patient. The relatively narrow biliary tract ducts consist of a series of bifurcations linking the liver, gallbladder, and pancreas via the papilla to the duodenal space for the transportation of bile and related enzymic substances for many metabolic functions but most commonly the body's ability to digest and absorb fats and vitamins D and K. However, blockages may occur in the pancreaticobiliary tree due to tumor related duct narrowing, stricture formation, infections, or stone and sludge generation among other etiologies. Endoscopic retrograde cholangiopancreatography (ERCP) is used to diagnose and treat these duct narrowings, regardless of the malignant or benign nature of the disease. Commonly a fully covered self-expanding metal stent (SEMS) may be used such that the radial forces of the stent scaffold the stricture. However, occlusions may occur within the stent after stent placement. Occlusive events can be a combination of local factors such as, but not limited to, inadequate stricture resolution and/or irregular bile properties. Occlusion can also be related to stent design components such as, but not limited to, coating tackiness and/or an interaction between the wire of the stent and bile (both causing drag and pooling of bile which accumulates over a time course). Due to the remote nature of a placed SEMS, the ability to diagnose and interact with a potential occlusive event is limited and typically can only be resolved through intervention at a stage when the occlusive event has demonstrated an exacerbation of a patient's symptoms (e.g., jaundice, abdominal pain, etc.). Occlusion may result in increased procedural charges due to reintervention and stent replacement, patient discomfort even though the stent device itself may be functioning effectively as a scaffold, retarding of the vessel, and/or an unjustified reputation for particular device families as being less efficacious or brands spurned due to historical or anecdotal knowledge of occlusive or suggestive occlusive events. The present disclosure is directed towards alternative stent designs which offer opportunities for prophylactic or preventative intervention to restore the patency of stents without surgical intervention thereby increasing stent efficacy and/or extending an operational lifetime of the stent. While the present disclosure is described with respect to the pancreaticobiliary ductal system, the devices, systems, and/or methods described herein may be used in stents or endoluminal implants positioned in other parts of the body, such as, but not limited to, bodily tissue, bodily organs, vascular lumens, non-vascular lumens and combinations thereof, such as, but not limited to, in the coronary or peripheral vasculature, trachea, bronchi, colon, small intestine, esophagus, biliary tract, urinary tract, prostate, brain, stomach, and the like.

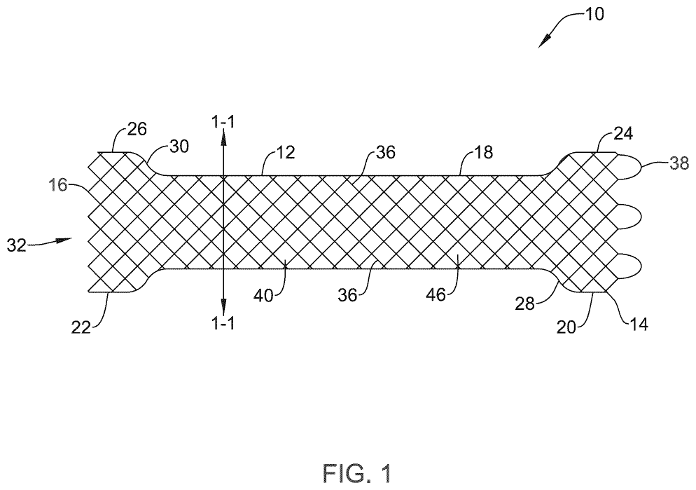

FIG. 1 illustrates a side view of an illustrative endoluminal implant 10, such as, but not limited to, a stent. In some instances, the stent 10 may be formed from an elongated tubular member 12. While the stent 10 is described as generally tubular, it is contemplated that the stent 10 may take any cross-sectional shape desired. The stent 10 may have a first, or proximal end 14, a second, or distal end 16, and an intermediate region 18 disposed between the first end 14 and the second end 16. The stent 10 may include a lumen 32 extending from a first opening adjacent the first end 14 to a second opening adjacent to the second end 16 to allow for the passage of food, fluids, etc.

The stent 10 may be expandable from a first radially collapsed configuration (not explicitly shown) to a second radially expanded configuration. In some cases, the stent 10 may be deployed to a configuration between the collapsed configuration and a fully expanded configuration. The stent 10 may be structured to extend across a stricture and to apply a radially outward pressure to the stricture in a lumen to open the lumen and allow for the passage of substances.

In some embodiments, the proximal end 14 of the stent 10 may include a plurality of loops 38. The loops 38 may be configured to receive a retrieval tether or suture (not explicitly shown) interwoven therethrough, or otherwise passing through one or more of the loops 38. The retrieval suture may be used to collapse and retrieve the stent 10, if so desired. For example, the retrieval suture may be pulled like a drawstring to radially collapse the proximal end 14 of the stent 10 to facilitate removal of the stent 10 from a body lumen.

The stent 10 may have a woven structure, fabricated from a number of filaments or struts 36 forming a tubular wall. In some embodiments, the stent 10 may be knitted or braided with a single filament or strut interwoven with itself and defining open cells 46 extending through the thickness of the tubular wall of the stent 10. In other embodiments, the stent 10 may be braided with several filaments or struts interwoven together and defining open cells 46 extending along a length and around the circumference of the tubular wall of the stent 10. The open cells 46 may each define an opening from an outer surface of the tubular wall to an inner surface of the tubular wall (e.g., through a thickness thereof) that is free from the filaments or struts 36. Some exemplary stents including braided filaments include the WallFlex®, WALLSTENT®, and Polyflex® stents, made and distributed by Boston Scientific, Corporation. In another embodiment, the stent 10 may be knitted, such as the Ultraflex™ stents made by Boston Scientific, Corporation. In yet another embodiment, the stent 10 may be of a knotted type, such as the Precision Colonic™ stents made by Boston Scientific, Corporation. In still another embodiment, the stent 10 may be a laser cut tubular member, such as the EPIC™ stents made by Boston Scientific, Corporation. A laser cut tubular member may have an open and/or closed cell geometry including one or more interconnected monolithic filaments or struts defining open cells 46 therebetween, with the open cells 46 extending along a length and around the circumference of the tubular wall. The open cells 46 may each define an opening from an outer surface of the tubular wall to an inner surface of the tubular wall (e.g., through a thickness thereof) that is free from the interconnected monolithic filaments or struts. In some instances, an inner and/or outer surface of the tubular wall of the stent 10 may be entirely, substantially, or partially, covered with a polymer coating 40, as will be described in more detail herein. The polymer coating 40 may extend across and/or occlude one or more, or a plurality of the cells 46 defined by the filaments or struts 36. In some cases, the stent 10 may be a self-expanding stent (SES), although this is not required.

In some instances, in the radially expanded configuration, the stent 10 may include a first end region 20 proximate the proximal end 14 and a second end region 22 proximate the second end 16. In some embodiments, the first end region 20 and the second end region 22 may include retention features or anti-migration flared regions 24, 26 having enlarged diameters relative to the intermediate region 18. The anti-migration flared regions 24, 26, which may be positioned adjacent to the first end 14 and the second end 16 of the stent 10, may be configured to engage an interior portion of the walls of the body lumen. In some embodiments, the retention features, or flared regions 24, 26 may have a larger diameter than the cylindrical intermediate region 18 of the stent 10 to prevent the stent 10 from migrating once placed in the body lumen. It is contemplated that the transition 28, 30 from the cross-sectional area of the intermediate region 18 to the retention features or flared regions 24, 26 may be gradual, sloped, or occur in an abrupt step-wise manner, as desired.

In some embodiments, the first anti-migration flared region 24 may have a first outer diameter and the second anti-migration flared region 26 may have a second outer diameter. In some instances, the first and second outer diameters may be approximately the same, while in other instances, the first and second outer diameters may be different. In some embodiments, the stent 10 may include only one or none of the anti-migration flared regions 24, 26. For example, the first end region 20 may include an anti-migration flare 24 while the second end region 22 may have an outer diameter similar to the intermediate region 18. It is further contemplated that the second end region 22 may include an anti-migration flare 26 while the first end region 20 may have an outer diameter similar to an outer diameter of the intermediate region 18. In some embodiments, the stent 10 may have a uniform outer diameter from the first end 14 to the second end 16. It is contemplated that the outer diameter of the stent 10 may be varied to suit the desired application.

It is contemplated that the elongated tubular member of the stent 10 can be made from a number of different materials such as, but not limited to, metals, metal alloys, shape memory alloys, and/or polymers, as desired, enabling the stent 10 to be expanded into shape when accurately positioned within the body. In some instances, the material may be selected to enable the stent 10 to be removed with relative ease as well. For example, the elongated tubular member of the stent 10 can be formed from alloys such as, but not limited to, nitinol and Elgiloy®. Depending on the material selected for construction, the stent 10 may be self-expanding or require an external force to expand the stent 10. In some embodiments, composite filaments may be used to make the stent 10, which may include, for example, an outer shell or cladding made of nitinol and a core formed of platinum or other radiopaque material. It is further contemplated the elongated tubular member of the stent 10 may be formed from polymers including, but not limited to, polyethylene terephthalate (PET). In some instances, the filaments of the stent 10, or portions thereof, may be bioabsorbable or biodegradable, while in other instances the filaments of the stent 10, or portions thereof, may be biostable.

As described above, the inner and/or outer surface of the tubular wall of the stent 10 may be entirely, substantially, or partially covered with a polymer coating 40. The polymer coating 40 may be silicone, polyurethane, or other flexible polymeric material. The polymer coating 40 may be generally taut and extend in the same plane as the struts 36. The coating can be applied as described herein.

The polymer coating 40 may span or be disposed within openings or interstices 46 defined between adjacent stent filaments or struts 36 of the scaffold structure. For example, the polymer coating may extend into the openings 46 defined between adjacent stent struts 36 and form an interface region. Further, the polymer coating 40 may additionally extend between adjacent filaments or struts 36, thereby filling any space between adjacent filaments or strut members 36, and thus prevent tissue ingrowth into the lumen of the stent 10.

The stent 10 can include magnetic nanoparticles (not shown in FIG. 1) that are disposed on or within one or more thermal-responsive layers on at least a portion of the stent 10, as described herein. In some examples, the magnetic nanoparticles may be arranged to suit a particular condition under treatment or the perceived location of potential occlusion. For example, if an occlusion is predicted to potentially occur higher in the biliary duct 100 (e.g., potentially due to stones, debris, etc.), the magnetic nanoparticles 48 may be located closer to the proximal end 14 of the stent 10. In another example, if an occlusion is predicted to potentially occur at the lower aspect of the stent 10 (e.g., potentially due to food impaction, etc.), the magnetic nanoparticles 48 may be located closer to the distal end 16 of the stent 10 (e.g., near the duodenal end).

The magnetic nanoparticles 48 may be arranged in any uniform or non-uniform arrangement, as desired. In some cases, the magnetic nanoparticles 48 may be discrete elements. In yet other cases, the magnetic nanoparticles may substantially cover a portion of the stent 10. For instance, the magnetic nanoparticles 48 may be arranged in a number of different patterns such that the magnetic nanoparticles 48 extend over a length and/or circumference of the stent 10. For example, the magnetic nanoparticles 48 can be arranged in a hexagonal array (e.g., a uniformly distributed hexagonal array), among other possibilities. Arranging the magnetic nanoparticles in a uniform hexagonal array can promote uniform heating of the magnetic nanoparticles to an elevated temperature and thus can promote the uniform liquification of the thermo-responsive layer in which the magnetic nanoparticles arranged in the uniform hexagonal array.

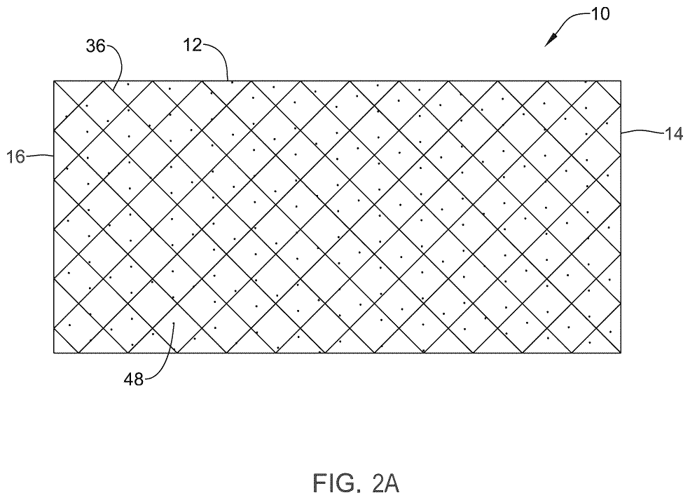

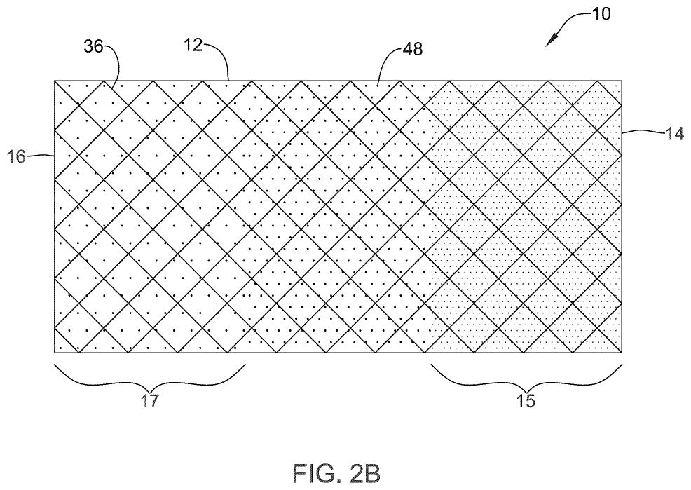

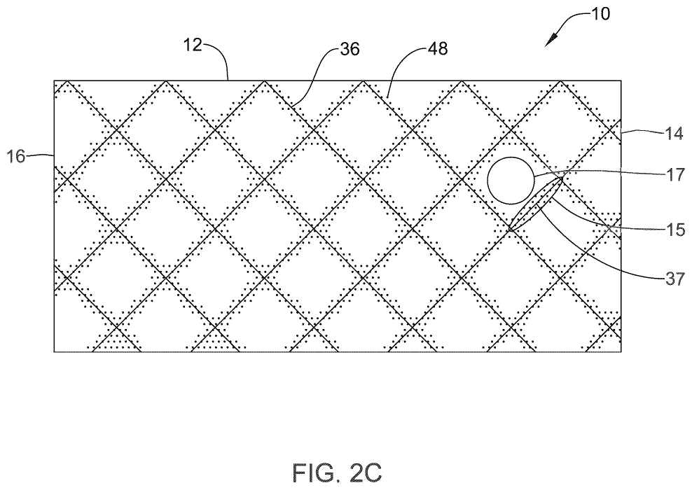

While FIG. 2A illustrates the magnetic nanoparticles 48 as generally uniformly distributed about a length of the stent 10 in a hexagonal array, being uniformly distributed or being in a hexagonal array is not required. Rather, in another example, the magnetic nanoparticles 48 may be arranged in one or more longitudinal arrays, one or more circumferential arrays, one or more helical arrays, or otherwise positioned on portions of the stent 10. The longitudinal arrays may be evenly or eccentrically spaced about a circumference of the stent 10 and may be spaced about an entirety of the circumference or less than an entirety of the circumference of the stent 10. Further, the stent 10 may include only a single array or more than one array of magnetic nanoparticles 48, as desired. The longitudinally extending arrays may extend along an entirety of a length of the stent 10 or less than an entirety of the length of the stent 10, as desired. The longitudinal arrays need not extend continuously along a length of the stent 10. For example, the arrays may include a gap or space therein.

In another example, the magnetic nanoparticles 48 may be arranged in one or more circumferential arrays. The circumferential arrays may be evenly or eccentrically spaced about a length of the stent 10 and may be spaced about an entirety of the length or less than an entirety of the length of the stent 10. Further, the stent 10 may include only a single array or more than one array of magnetic nanoparticles 48, as desired. The circumferentially extending arrays may extend about an entirety of a circumference of the stent 10 or less than an entirety of the circumference of the stent 10, as desired. The circumferentially extending arrays need not extend continuously along a circumference of the stent 10. For example, the circumferentially extending arrays may include a gap or a space therein.

In another example, the magnetic nanoparticles 48 may be arranged in one or more helical arrays. The helical arrays may be evenly or eccentrically spaced about a length of the stent 10 and may be spaced or extend along an entirety of the length and/or circumference or less than an entirety of the length and/or circumference of the stent 10. Further, the stent 10 may include only a single array or more than one array of magnetic nanoparticles 48, as desired. The helically extending arrays need not extend continuously along a length and/or circumference of the stent 10. For example, the helically extending arrays may include a gap or a space therein.

It is further contemplated that the magnetic nanoparticles 48 may be clustered at the proximal end 14 of the stent 10 and/or at the distal end 16 of the stent 10 while the intermediate region 18 is free from magnetic nanoparticles 48, in some embodiments. In yet other examples, the intermediate region 18 may include magnetic nanoparticles 48 while the proximal end 14 and/or distal end 16 of the stent 10 are free from magnetic nanoparticles 48. These are just some examples of potential arrangements of the magnetic nanoparticles 48. It should be understood that the magnetic nanoparticles 48 may be arranged in any arrangement, regular or irregular, as desired.

In some examples, the magnetic nanoparticles can be a ferromagnetic material such as iron (II/III) oxide. For instance, the magnetic nanoparticles 48 may be a colloid, such as, but not limited to, a silicone base having a quantity of ferromagnetic material, or other magnetic material, mixed therein. Other ferromagnetic materials may include, but are not limited to, cobalt nickel, rare-earth metals, and/or alloys or compounds thereof. It is contemplated that other types of magnetic materials may also be used, such as, but not limited to, ferrimagnetic, etc. The colloid may be combined using a standard mixing process. Iron oxide such as iron oxide powder can be added in various quantities to the silicone base to tailor the magnetic properties of the magnetic nanoparticles 48. It is contemplated that the colloid coating may be applied via dip coating, spray coating, or otherwise applied.

A variety of coating techniques may allow the magnetic nanoparticles 48 to be applied to very specific areas of the stent 10 in any pattern desired to achieve a desired effect. In some instances, the magnetic nanoparticles 48 may be secured to the thermo-responsive layer 52, while the magnetic nanoparticles 48 are spaced away from and/or not in direct contact with the struts 36 of the stent 10. In some embodiments, the magnetic nanoparticles 48 may be embedded (e.g., encapsulated) within the thermo-responsive layer 52 and spaced a distance away from the polymer coating 40 and any other adjacent thermo-responsive layer. However, this is not required, in some instances, a portion of the magnetic nanoparticles may extend axially a distance out of a thermo-responsive layer, for instance, into another adjacent thermo-responsive layer if present. In some embodiments, the magnetic nanoparticles 48 may be a ferromagnetic material that is embedded in another location of the stent 10 such as being embedded in the polymer coating 40 (e.g., silicon sheathing material) of the stent 10. For instance, the magnetic nanoparticles 48 may embedded exclusively in the polymer coating 40 (e.g., silicon sheathing material) of the stent 10. Having the magnetic nanoparticles 48 embedded exclusively in the polymer coating 40 may alleviate any concern associated with dispersion of the magnetic nanoparticles into the patient's body (e.g., responsive to application of a magnetic field and liquification of the thermo-responsive layer 52). Yet, in some embodiments the magnetic nanoparticles 48 may embedded in the polymer coating 40 and the thermo-responsive layer 52. Having the magnetic nanoparticles 48 embedded in the polymer coating 40 and the thermo-responsive layer 52 may increase a percentage of the thermo-responsive layer 52 that is liquified responsive to application of a magnetic field and/or may reduce an amount of time that the magnetic field is applied to the stent 10 to liquefy the thermo-responsive layer 52.

In some embodiments, the magnetic nanoparticles can have a number-average particle diameter in a range from about 1 nanometer to about 400 nanometers. All individual values and sub-ranges from 1 nanometers to 400 nanometers are included. For instance, the magnetic nanoparticles can have a number-average particle diameter in a range from 10 to 400 nanometers, from 20 to 350 nanometers, from 30 to 300 nanometers, from 20 to 200 nanometers, from 30 to 100 nanometers, from 30 to 80 nanometers, from 30 to 60 nanometers, from 40 to 60 nanometers, from 1 to 100 nanometers, or from 5 to 400 nanometers, among other possibilities. In some embodiments, each of the magnetic nanoparticles can have a number-average particle diameter of about 1 nanometer, about 2 nanometers, about 5 nanometers, about 10 nanometers, about 20 nanometers, about 30 nanometers, about 40 nanometers, about 50 nanometers, about 60 nanometers, about 80 nanometers, about 100 nanometers, about 150 nanometers, about 200 nanometers, about 250 nanometers, about 300 nanometers, about 350 nanometers, or about 400 nanometers. As mentioned, the term “about” generally refers to a range of numbers that one of skill in the art would consider equivalent to the recited value (i.e., having the same function or result). In many instances, the term “about” may be indicative as including numbers that are rounded to the nearest significant figure. For instance, in the context of number-average particle diameter, the term “about” may refer to values that are +/−5 percent, +/−10 percent, +/−15 percent, or +/−20 percent of a recited value. As used herein, the number-average particle diameter and distribution are determined in accordance with ASTM E2490-09 for nanoparticles up to 100 nm in diameter or transmission electron microscopy (TEM) for nanoparticles greater than 100 nm in diameter. Employing magnetic nanoparticles that have a number-average particle diameter in a range from 1 nanometer to 400 nanometers can promote aspects herein, such as promoting the magnetic nanoparticles to be excited to an elevated temperature in response to an applied magnetic field, promoting liquification of the thermo-responsive layer, promoting the thermo-responsive layer to mechanically decouple from a remaining portion of the thermo-responsive layer, a polymer coating, or both, and/or permitting the decoupled thermo-responsive layer to readily pass through and readily be eluted or expelled from a body of a patient. That is, in contrast to other approaches (e.g., those which employ stents which are intended to require direct mechanical removal (e.g., invasive surgery to remove an occluded stent), the approaches herein can restore stent patency in the absence of a direct mechanical intervention (e.g., in the absence of invasive surgery) due at least in part to the particular combination of materials employed herein (e.g., the thermo-responsive layer which liquefies and remains liquid (e.g., responsive to the cessation of an application of magnetic radiation) while in the body of the patent and/or the magnetic nanoparticles, etc.), as described herein. In some embodiments, the magnetic nanoparticles are configured to be excited to an elevated temperature that is a threshold amount (e.g., 3, degrees Celsius 4 degrees Celsius, or 5 degrees Celsius, etc.) higher than a physiological temperature (e.g., 37 degrees Celsius), as detailed herein. Thus, the thermo-responsive layer can be selectively liquified responsive to the application of magnetic radiation and may not inadvertently undergo a state change (e.g., may not transition from solid to liquid at the physiological temperature).

The magnetic nanoparticles can be uniformly dispersed on or embedded within the thermo-responsive layer or may be non-uniformly dispersed on or embedded within the thermo-responsive layer. For instance, FIG. 2A-2C illustrate side views of a portion of the illustrative endoluminal implant or stent having a thermo-responsive layer including magnetic nanoparticles in several illustrative arrangements.

FIG. 2A illustrates a side view of the illustrative endoluminal implant or stent having a thermo-responsive layer including magnetic nanoparticles 48 that are uniformly dispersed on or embedded within the thermo-responsive layer along a length of the thermo-responsive layer. Thus, in some embodiments the magnetic nanoparticles 48 can be uniformly dispersed on or embedded within the thermo-responsive layer along a portion of or along an entire length of the elongated tubular member. For instance, in some embodiments the magnetic nanoparticles 48 can be uniformly dispersed on or embedded within the thermo-responsive layer that extends along an entire length of the elongated tubular member. For example, that magnetic nanoparticles 48 can be uniformly dispersed on or embedded within the thermo-responsive layer in a hexagonal pattern extending along at least a portion of the length of the thermo-responsive layer, as illustrated in FIG. 2A. In embodiments with two or more thermo-responsive layers, the magnetic nanoparticles can be uniformly dispersed on or embedded within each thermo-responsive layer along a portion or along an entire length of the elongated tubular member. Having the magnetic nanoparticles be uniformly dispersed on or embedded within a thermo-responsive layer along a portion of or along an entire length of the elongated tubular member can promote aspects herein, such as promoting one or more magnetic nanoparticles to be excited to an elevated temperature by application of a magnetic field and/or promoting the uniform liquification and mechanically decoupling of the thermo-responsive layer from a remaining portion of the thermo-responsive layer, the polymer coating, or both. In some instances, each of the magnetic nanoparticles that are uniformly dispersed on or embedded within the thermo-responsive layer can be the same type, same shape, and same size of magnetic nanoparticles (e.g., when accounting for any differences that are attributable to manufacturing tolerances). However, in some instances, one or more of the magnetic nanoparticles that are uniformly dispersed on or embedded within the thermo-responsive layer can be different type, different shape, and/or different size of magnetic nanoparticle.

Having the magnetic nanoparticles be uniformly dispersed on or within a thermo-responsive layer is not required. Rather, as illustrated in FIG. 2B and FIG. 2C, in some cases the magnetic nanoparticles 48 can be non-uniformly dispersed on or embedded within a thermo-responsive layer. Being non-uniformly dispersed refers to having different concentrations of magnetic nanoparticles 48 in different regions along at least a portion of a length or circumference of an elongated tubular member where the magnetic nanoparticles are disposed. In some instances, each of the magnetic nanoparticles that are non-uniformly dispersed on or embedded within the thermo-responsive layer can be the same type, same shape, and same size of magnetic nanoparticles (e.g., when accounting for any differences that are attributable to manufacturing tolerances). However, in some instances, one or more of the magnetic nanoparticles that are non-uniformly dispersed on or embedded within the thermo-responsive layer can be different type, different shape, and/or different size of magnetic nanoparticle.

For instance, FIG. 2B illustrates a side view of the illustrative endoluminal implant or stent having a thermo-responsive layer including magnetic nanoparticles 48 arranged in a gradient extending from a first region 15 to a second region 17. As illustrated in FIG. 2B, the gradient can have a higher concentration of the magnetic nanoparticles 48 in the first region 15 adjacent to a first end of the elongated tubular member, relative to a concentration of the magnetic nanoparticles 48 in the second region 17 adjacent to a second end of the elongated tubular member 12. In some embodiments, the first end of the elongated tubular member can refer to the proximal end 14 of the elongated tubular member 12 and the second end of the elongated tubular member can refer to the distal end 16 of the elongated tubular member. However, in some embodiments, the first end can refer to the distal end 16 of the elongated tubular member 12 and the second end can refer to the proximal end of the elongated tubular member 12. The magnetic nanoparticles can be arranged in a gradient that extends along a portion of or an entire length of the thermo-responsive layer. For instance, in some embodiments, the magnetic nanoparticles can be arranged in a gradient that extends along an entire length of the thermo-responsive layer. Having the magnetic nanoparticles arranged in a gradient extending along a portion or along an entire length of the elongated tubular member 12 can promote aspects herein, such as promoting one or more magnetic nanoparticles to be excited to an elevated temperature by application of a magnetic field and/or promoting the selective or expediated liquification and mechanically decoupling of a portion of the thermo-responsive layer from a remaining portion of the thermo-responsive layer, the polymer coating, or both. For instance, employing a gradient extending from a first end to a second end of the elongated tubular member having a higher concentration of magnetic nanoparticles adjacent to the first end than a concentration of magnetic nanoparticles adjacent to the second end of the elongated tubular member may promote the selective or expediated liquefication and mechanically decoupling of a first portion of the thermo-responsive layer at the first end of the elongated tubular member, relative to any liquification and mechanical decoupling of a second portion of thermo-responsive layer at the second end of the elongated tubular member.

FIG. 2C illustrates a side view of the illustrative endoluminal implant or stent having a thermo-responsive layer including magnetic nanoparticles dispersed with a higher concentration of magnetic nanoparticles adjacent to structures (e.g., struts) forming the tubular wall. As illustrated in FIG. 2C, the regions (e.g., a first region 37) with higher concentrations of magnetic nanoparticles in the thermo-responsive layer can be adjacent to or more proximate to each of the structures forming the tubular wall. For instance, the regions with higher concentrations of magnetic nanoparticles in the thermo-responsive layer can be adjacent to each of the structures forming the tubular wall along an entire length of the tubular wall. In some embodiments, the structures forming the tubular wall can be struts, among other possibilities. Having the magnetic nanoparticles be dispersed with a higher concentration of magnetic nanoparticles adjacent to structures (e.g., struts) forming the tubular wall can promote aspects herein, such as promoting one or more magnetic nanoparticles to be excited to an elevated temperature by application of a magnetic field and/or promoting expediated liquification and mechanically decoupling of at least a portion of the thermo-responsive layer from a remaining portion of the thermo-responsive layer, the polymer coating, or both. For instance, employing magnetic nanoparticles dispersed with a higher concentration of magnetic nanoparticles adjacent to structures (e.g., struts) forming the tubular wall can promote the liquefaction of the thermo-responsive layer that is proximate to the structures and thereby promote the selective liquefication and mechanically decoupling of at least a portion of the thermo-responsive layer that is proximate to the structures, while employing a relatively small amount of magnetic nanoparticles. For instance, in some embodiments magnetic nanoparticles may be disposed only in the first region 37 is adjacent to one or more structures forming the tubular wall, and the magnetic nanoparticles may not be disposed in a second region 17 that is less proximate to the one or more structures forming the tubular wall.

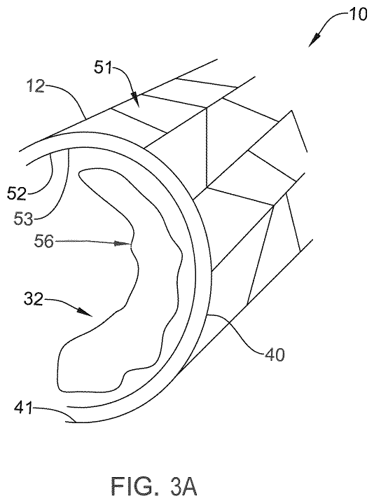

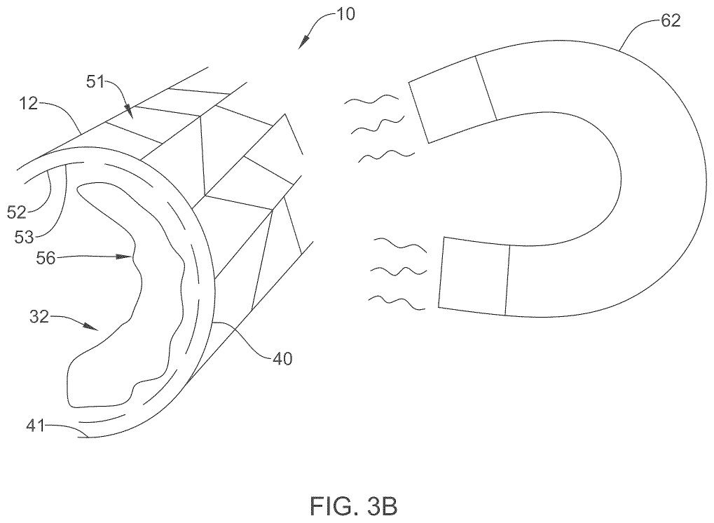

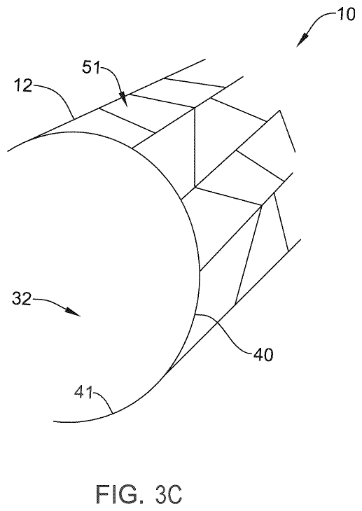

FIG. 3A is a schematic cross-sectional view of the endoluminal implant or illustrative stent 10 having a thermo-responsive layer including magnetic nanoparticles (not illustrated in FIG. 3A) prior to magnetization, taken at line 1-1 of FIG. 1. FIG. 3B is a schematic cross-sectional view of the illustrative endoluminal implant or stent 10 during magnetization. FIG. 3C is a schematic cross-sectional view of the illustrative endoluminal implant or stent 10 after magnetization.

As illustrated in FIGS. 3A-3C, the stent 10 can includes the elongated tubular member 12 forming a tubular wall 51. The tubular wall 51 can extend along at least a portion of the length of the stent 10 between a distal end to a proximal end of the stent 10. For instance, the tubular wall 51 can have a length that is at least equal to a cylindrical intermediate region (e.g., the cylindrical intermediate region 18, as illustrated in FIG. 1) of the stent 10. In some embodiments, the tubular wall 51 can extend an entire length of the stent 10 between a distal end and a proximal end of the stent 10.

The stent 10 can include a polymer coating 40 disposed on a surface of the tubular wall 51. For instance, the polymer coating 40 can be disposed at least on an inner surface of the tubular wall 51. The polymer coating 40 can be disposed along at least a portion of the length and at least a portion of a circumference of the tubular wall 51. In some embodiments, the polymer coating 40 can extend from a proximal end to a distal end of the elongated tubular member 12. For instance, the polymer coating 40 can be disposed along an entire length and around an entire circumference of the tubular wall 51, where the tubular wall extends at least a portion of or an entire length between a distal end and a proximal end of the stent 10. In some embodiments, the polymer coating 40 includes silicone or polyurethane.

The stent 10 can include a thermo-responsive layer 52 disposed at least on an inner surface 41 of the polymer coating 40. For instance, the thermo-responsive layer 52 can be disposed only on an inner surface of the polymer coating 40. In some embodiments, the thermo-responsive layer 52 can be disposed directly on (without intervening layers or elements) the inner surface 41 of the polymer coating 40. The thermo-responsive layer 52 can be disposed along at least a portion of the length and along at least a portion of a circumference of the polymer coating 40. In some embodiments, the thermo-responsive layer 52 can extend from the proximal end to the distal end of the elongated tubular member. In some embodiments, the thermo-responsive layer 52 can be disposed along an entire length and around an entire circumference of the polymer coating 40.

In some embodiments, the thermo-responsive layer 52 can include a thermo-responsive polymer or a thermo-responsive hydrogel. In some cases, the thermo-responsive layer 52 can be formed of a bio-degradable, bio-inert, and/or non-toxic thermo-responsive polymer or a bio-degradable, bio-inert, and/or non-toxic thermo-responsive hydrogel. For instance, the thermo-responsive layer 52 can be formed of a collagen and polycaprolactone matrix such as a collagen gel and a polycaprolactone film matrix. Employing a collagen and polycaprolactone matrix can yield a thermo-responsive layer with good mechanical stability, is thermally irreversible (e.g., transitions from a solid state to a liquid state at an elevated temperature responsive to the application of a magnetic field and remains liquid when the field is removed/discontinued), and yet is bio-degradable and non-toxic. For instance, the collagen and polycaprolactone matrix can be configured to have a sharp phase transition (e.g., occurring over 3 degrees Celsius, 4 degrees Celsius, or 5 degrees Celsius). The collagen and polycaprolactone matrix can be configured to have a sharp phase transition by varying a collagen to polycaprolactone ratio in the collagen and polycaprolactone matrix and/or by including additional or alternate components such as including fillers and/or other thermally-sensitive polymers and/or hydrogels.

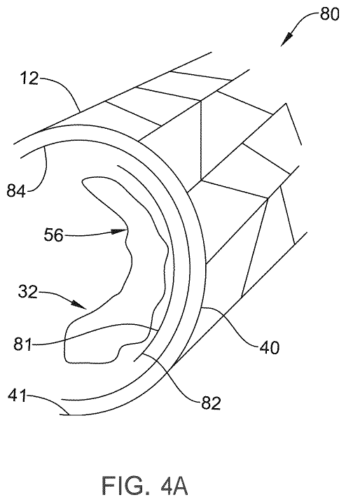

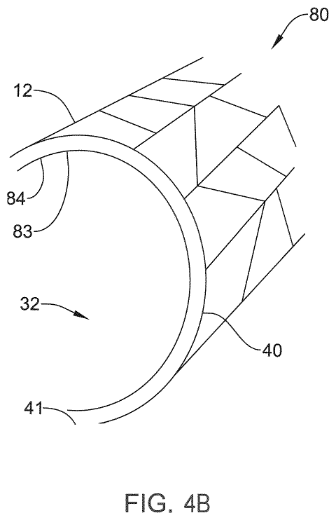

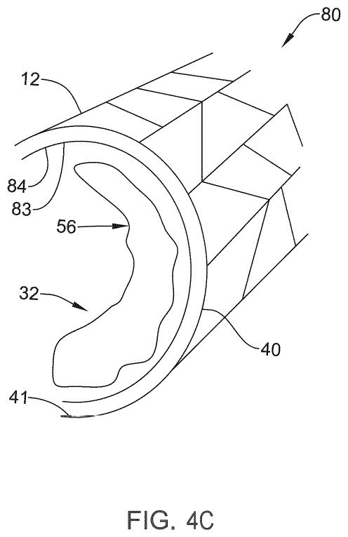

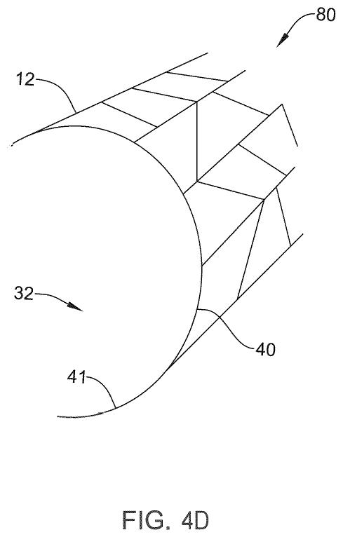

In some embodiments, the thermo-responsive layer can be an individual thermo-responsive layer such as the thermo-responsive layer 52, as illustrated in FIG. 3A. However, in some embodiments the thermo-responsive layer can include a plurality of thermo-responsive layers (e.g., having differing amounts and/or arrangements or sizes of magnetic nanoparticles), as described herein with respect to FIGS. 4A-4D.

As illustrated in FIG. 3A, a lumen 32 of the stent 10 can become at least partially occluded by material such as biliary sludge 56. For instance, over time the biliary sludge 56 may adhere to an inner surface 53 of the thermo-responsive layer 52 of the stent 10. The biliary sludge 56 may extend radially from the inner surface 53 of the thermo-responsive layer 52 into the lumen 32, thereby blocking at least a portion of a flow through the lumen 32 and thus may inhibit efficacy of the stent 10.

As mentioned, the thermo-responsive layer 52 can include magnetic nanoparticles (not illustrated in FIG. 3A-3C) disposed on or within the thermo-responsive layer 52. At least a portion of the thermo-responsive layer 52 can be configured to liquify and mechanically decouple from a remaining portion of the thermo-responsive layer 52, the polymer coating 40, or both. For instance, the entire thermo-responsive layer 52 (e.g., along the entire circumference of the inner surface 41 of the polymer coating 40) can be configured to liquify and mechanically decoupled from the inner surface 41 of the polymer coating 40 in response to application of a magnetic field that is external to a patient's body. As illustrated in FIG. 3B, the application of the external magnetic field (represented as element 62) can cause the liquification and mechanical decoupling of at least a portion of the thermo-responsive layer 52 from the inner surface 41 of the polymer coating 40. Application of the magnetic field can continue until some or all of the thermo-responsive layer 52 is liquified and mechanically decoupled from the inner surface 41 of the polymer coating 40. For instance, as illustrated in FIG. 3C the entire thermo-responsive layer 52 can be liquified and mechanically decoupled from the inner surface 41 of the polymer coating 40. The selective liquefaction and mechanically decoupling of at least a portion of the thermo-responsive layer 52 responsive to the application of an external magnetic field can restore the patency of the stent 10. Namely, the biliary sludge 56 that was previously coupled to at least a portion of the inner surface 53 of the thermo-responsive layer 52 along with the liquified thermo-responsive layer 52 corresponding to the at least the portion of the inner surface 53 to which the biliary sludge 56 was adhered can be dislodged from the stent 10. Thus, a diameter of the lumen 32 can be restored to substantially biliary sludge-free diameter without surgical intervention, thereby increasing stent efficacy and/or an operational lifetime of the stent. The polymer coating 40 can remain intact during and following the selective liquification of the thermo-responsive layer 52. For instance, the entire polymer coating layer 40 can remain intact during and following the selective liquification of the thermo-responsive layer 52. The presence of the intact polymer layer 40 can promote aspects herein, such as mitigating tissue in-growth into the lumen 32.

FIG. 4A is a schematic cross-sectional view of an illustrative endoluminal implant or stent 80 having a plurality of thermo-responsive layers including magnetic nanoparticles prior to magnetization, taken at line 1-1 of FIG. 1. FIG. 4B is a schematic cross-sectional view of the illustrative endoluminal implant or stent after magnetization at a first frequency. FIG. 4C is a schematic cross-sectional view of the illustrative endoluminal implant or stent a period of time following the magnetization at the first frequency. FIG. 4D is a schematic cross-sectional view of the illustrative endoluminal implant or stent after magnetization at a second frequency. The stent 80 illustrated in FIGS. 4A-4D is analogous to the stent 10 illustrated in FIG. 3A-3C, with the difference that the stent 80 includes a plurality of thermo-responsive layers rather than an individual thermo-responsive layer. In some embodiments, the second thermo-responsive layer 84 can be disposed directly on (without intervening layers or elements) and extend around an entire circumference of the inner surface 41 of the polymer coating 40 and the first thermo-responsive layer 82 can be disposed directly disposed on and extend around an entire circumference of the inner surface 83 of the second thermo-responsive layer 84.

The stent 80 can include the elongated tubular member 12 forming a tubular wall 51 and a polymer coating 40 disposed on at least on an inner surface of the tubular wall 51. The stent 80 includes a plurality of thermo-responsive layers. For instance, the stent can include two thermo-responsive layers such as a first thermo-responsive layer 82 and a second thermo-responsive layer 84. The second thermo-responsive layer 84 can be disposed on an inner surface 41 of the polymer coating 40 and a first thermo-responsive layer 82 can be disposed on an inner surface 83 of the second thermo-responsive layer 84. As illustrated in FIG. 4A, the second thermo-responsive layer 84 can be disposed directly on (without intervening layers or elements) an inner surface 41 of the polymer coating 40 and the first thermo-responsive layer 82 can be directly disposed on an inner surface 83 of the second thermo-responsive layer 84.

The second thermo-responsive layer 84 (e.g., an inner thermo-responsive layer) can be configured to be excited to a second elevated temperature in response to an applied magnetic field having a second frequency. The first thermo-responsive layer 82 (e.g., an outer thermo-responsive layer) can be configured to be excited to a first elevated temperature in response to an applied magnetic field having a first frequency. The first thermo-responsive layer 82 and the second thermo-responsive layer 84 can be configured to liquify at different elevated temperatures. For instance, the second thermo-responsive layer 84 can be configured to liquify responsive to at an elevated temperature that is higher than an elevated temperature at which the first thermo-responsive layer 82 is configured to liquify. Thus, as described herein, the first thermo-responsive layer 82 can be liquified and mechanically decoupled from the second thermo-responsive layer 84 which can remain intact during the application of magnetic radiation to the stent and liquification of the first thermo-responsive layer 82. In this way, the stent 80 can undergo a plurality of applications of magnetic radiation to iteratively liquify some of the plurality of thermo-responsive layers, thereby permitting restoring patency of the stent 80 a plurality of times. For instance, the stent 80 can undergo the application of magnetic radiation at in two different time periods (e.g., initially to liquify and remove the first thermo-responsive layer 82 and subsequently to liquify and remove the second thermo-responsive layer 84), and thereby restore the patency of the stent 80 on two separate occasions to increase stent efficacy and operation lifetime of the stent.

The first thermo-responsive layer 82 and the second thermo-responsive layer 84 may be formed as separate layers. The first thermo-responsive layer 82 and the second thermo-responsive layer 84 may be formed from the same material or different materials, as desired. In some embodiments, the first thermo-responsive layer 82 and the second thermo-responsive layer 84 can include different concentrations, types, sizes, and/or arrangements of magnetic nanoparticles. For instance, the second thermo-responsive layer 84 can include a lower concentration of magnetic nanoparticles and/or can include a different (e.g., smaller) size of nanoparticles than the first thermo-responsive layer 82 such that the first thermo-responsive layer 82 can be configured to be excited to a first elevated temperature in response to an applied magnetic field (e.g., having a first strength and/or frequency), while the second thermo-responsive layer 84 can be configured to be excited to a second elevated temperature in response to an applied magnetic field (e.g., having the second strength and/or frequency that is different than the first strength and/or frequency). However, in some embodiments, the first thermo-responsive layer 82 and the second thermo-responsive layer 84 can include the same concentration, type, size, and arrangements of magnetic nanoparticles, but can be formed of different types of materials (e.g., different polymers) such that the first thermo-responsive layer 82 can be configured to be excited to a first elevated temperature in response to an applied magnetic field, while the second thermo-responsive layer 84 can be configured to be excited to a second elevated temperature in response to an applied magnetic field.

In some embodiments, the magnetic field may be selectively applied along the length of the stent 80. In FIG. 4A, the stent 80 is in an initial state with no magnetic field being applied. As can be seen, the first thermo-responsive layer 82 and the second thermo-responsive layer 84 are each present in the stent 80. In the presence of the applied magnetic field at a first frequency, the magnetic nanoparticles disposed on or in the first thermo-responsive layer 82 are excited (e.g., undergo magnetic hyperthermia) to an elevated temperature (e.g., a first elevated temperature) such that the first thermo-responsive layer 82 is liquified and mechanically detaches from at least a portion of an inner surface 83 of the second thermo-responsive layer 84. As such, the biliary sludge 56 (illustrated in FIG. 4A as being attached to an inner surface 81 of the first thermo-responsive layer 82) can be removed from the lumen 32 of the stent 80. Thus, as illustrated in FIG. 4B, the first thermo-responsive layer 82 and the biliary sludge 56 are no longer present in the stent 80 following magnetization at the first frequency.

Subsequent to the liquification and mechanical removal of the first thermo-responsive layer 82 and the biliary sludge 56 initially present in the stent 80, additional biliary sludge may accumulate on an inner surface 83 of the second thermo-responsive layer 84, as the inner surface 83 is now being exposed to and in fluidic contact with material in the lumen 32. As illustrated in FIG. 4C, biliary sludge 56 has accumulated on the inner surface 83 of the second thermo-responsive layer 84 after period of time following the magnetization at the first frequency. In FIG. 4C, the stent 80 is in another initial state with no magnetic field being applied.

In the presence of the applied magnetic field, the magnetic nanoparticles in the second thermo-responsive layer 84 are excited (e.g., undergo magnetic hyperthermia) by magnetization at a second field strength and/or frequency to an elevated temperature (e.g., a second elevated temperature) at which the second thermo-responsive layer 84 is liquified and mechanically detaches from at least a portion of the inner surface 41 of the polymer coating 40. As such, the biliary sludge 56 (illustrated in FIG. 4C) can be removed from the lumen 32 of the stent 80. Thus, as illustrated in FIG. 4D, the first thermo-responsive layer 82, the second thermo-responsive layer 84, and the biliary sludge 56 (e.g., as illustrated in FIG. 4A and as illustrated in FIG. 4C) are no longer present in the stent 80 following magnetization at the second frequency. In some cases, the first frequency may be a lower frequency that the second frequency. However, in some instances, the first frequency can be equal to or greater than the second frequency. In some cases, the first frequency and the second frequency can be respective frequencies within a range from 100 kilohertz to 500 kilohertz.

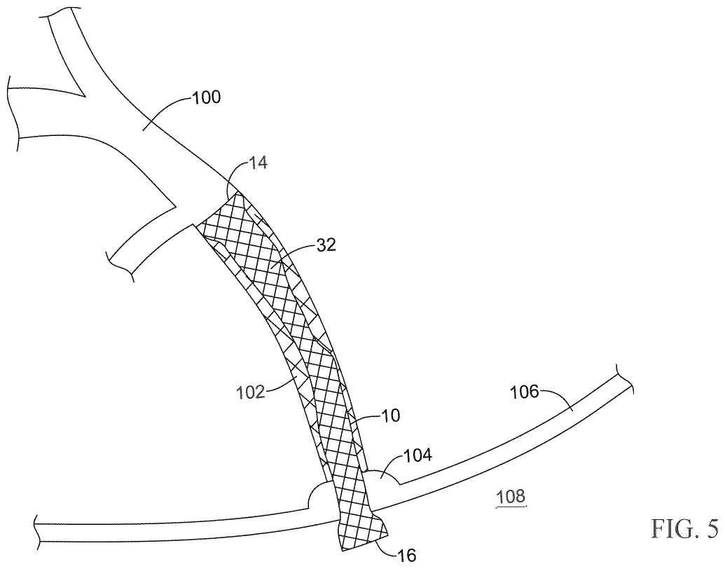

FIG. 5 depicts a schematic view of the illustrative stent 10 deployed within the biliary tree. In the illustrated example, the stent 10 is positioned within a biliary duct 100 and is positioned across a stricture 102. In some embodiments, an entirety of a stent may be exposed to the magnetic field at a same time. In yet other embodiments, the magnetic field may be applied to a region of the stent adjacent to the stricture 102 or other diseased site.

The distal end 16 of the stent 10 extends past the papillary mass 104, through the duodenal wall 106 and into the duodenum 108. The stent 10 may be positioned within the biliary tree via endoscopic retrograde cholangiopancreatography (ERCP). For example, the stent 10 may be guided to the biliary location and subsequently deployed, as shown in FIG. 5. The patient may be discharged with the stent 10 in-situ. For subsequent maintenance of the stent 10 (e.g., to restore patency of the stent 10 by resolving occlusion of the lumen 32 of the stent) after a period of time, the patient may be guided through a non-invasive treatment option. For example, the stent 10 may be subjected to a magnetic field to cause the magnetic nanoparticles disposed on or within a thermo-responsive layer of the stent 10 to be excited (e.g., undergo magnetic hyperthermia) to an elevated temperature.

In some embodiments, the magnetic nanoparticles are configured to be excited to an elevated temperature that is a threshold amount (e.g., 3, degrees Celsius 4 degrees Celsius, or 5 degrees Celsius, etc.) higher than a physiological temperature (e.g., 37 degrees Celsius). In some embodiments, the elevated temperature corresponds to (e.g., is equal to or greater than) a melting point or critical solution temperature (CST) of the thermo-responsive layer. Thus, the thermo-responsive layer can be selectively liquified responsive to the application of magnetic radiation which raises the temperature of the magnetic nanoparticles and the thermo-responsive layer to or above an elevated temperature, and yet the thermo-responsive layer may not inadvertently undergo liquification (e.g., responsive to being at a physiological temperature). For instance, in some embodiments, the magnetic nanoparticles are configured to be excited to an elevated temperature in a range from 42 degrees Celsius to 58 degrees Celsius, in a range from 42 degrees Celsius to 56 degrees Celsius, in a range from 44 degrees Celsius to 56 degrees Celsius, in a range from 44 degrees Celsius to 52 degrees Celsius, in a range from 46 degrees Celsius to 52 degrees Celsius, among other possibilities. Similarly, in some embodiments the thermo-responsive layer is configured with a melting point or critical solution temperature in the range from 42 degrees Celsius to 58 degrees Celsius, in a range from 42 degrees Celsius to 56 degrees Celsius, in a range from 44 degrees Celsius to 56 degrees Celsius, in a range from 44 degrees Celsius to 52 degrees Celsius, in a range from 46 degrees Celsius to 52 degrees Celsius, among other possibilities.

In some embodiments, the magnetic nanoparticles can be included in a plurality of thermo-responsive layers that are each configured to liquify at different respective elevated temperatures. For instance, a first thermo-responsive layer can include magnetic particles that are configured to be excited to a first elevated temperature (e.g., at which the first thermo-responsive layer undergoes liquification) and a second thermo-responsive layer can include magnetic particles that are configured to be excited to a second elevated temperature that is different than (e.g., less than) the first elevated temperature. In some cases, the first elevated temperature can be different than the second elevated temperature by a threshold amount (e.g., 1, 2, 3, 4, 5, or 6 degrees, etc.). For instance, in some cases that first elevated temperature can be equal to 46 degrees Celsius and the second elevated temperature can be equal to 52 degrees Celsius, among other possibilities.

As mentioned, at least a portion of the thermo-responsive layer can be configured to liquify (e.g., change from a solid state to a liquid state) in response to one or more magnetic nanoparticles being excited to or above an elevated temperature. The liquified portion of the thermo-responsive layer can be configured to mechanically decouple from a surface of a remaining portion of the thermo-responsive layer, a surface of a polymer coating on the implanted stent, or both. Thus, any biliary sludge coupled to the liquified portion of the thermo-responsive layer can be released from the stent. The thermo-responsive layer can be configured to remain liquified (e.g., responsive to the cessation of the application of the magnetic field) once exposed to the magnetic field and excited to the elevated temperature. For instance, the thermo-responsive layer can be configured to remain liquified while inside of a patient's body (e.g., while at or above a physiological temperature) so the liquified thermo-responsive layer, along with any released biliary sludge, can readily pass through and out of the patient's body. The selective liquification of at least a portion of the thermo-responsive layer can thereby restore patency of the lumen 32 of the stent 10. It is contemplated that the time period between applications of the magnetic field may vary depending on whether or not the patient is symptomatic (e.g., showing signs of an occlusion), among other factors. In some cases, the time period between implant of the stent and application of the magnetic field may be in the range of weeks, months, or years. In some cases, the time period between a first application of the magnetic field (e.g., at a first frequency) and a second application of the magnetic field (e.g., at a second frequency that is different than the first frequency) may be in the range of weeks, months, or years. It is further contemplated that the magnetic field may be applied at an outpatient facility, at a step-down facility, or as a home care option with the correct instructions. The application of a magnetic field to the stent 10 may be a less invasive and less costly intervention compared to surgical correction of an occlusion. For instance, the magnetic field is applied exterior of the patient's body and therefore is a less invasive and less costly intervention compared to surgical correction of an occlusion.

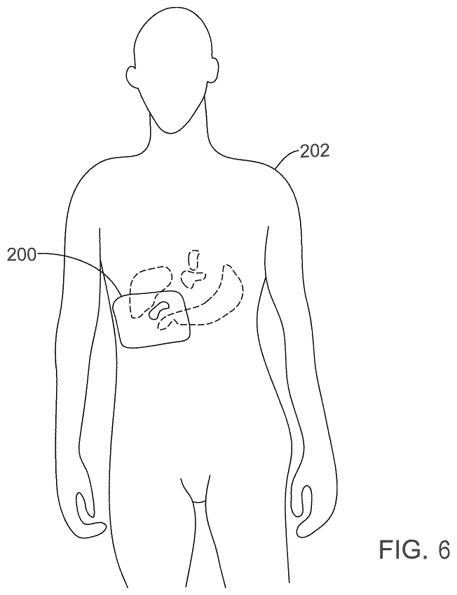

FIG. 6 illustrates a schematic view of the illustrative placement of a console 200 for generating a magnetic field relative to the body of the patient 202. The console 200 is positioned exterior to the patient 202 adjacent to the desired treatment region. In FIG. 6, the console 200 is positioned adjacent to the biliary tree. It is contemplated that the console 200 may be positioned at any location of the patient 202 to achieve the desired treatment, including, but not limited to the, the anterior side, the posterior side, the left side, or the right side, etc. In some cases, the console 200 can represent a magnetic resonance imaging (MRI) device.

The magnetic field oscillating frequency in either the alternating magnetic field or the pulsed magnetic field may be controlled and adjusted via the console 200. For example, the frequency may be adjusted to a frequency (e.g., a first frequency) associated with a first thermo-responsive layer or may be adjusted to a frequency (e.g., a second frequency) associated with a second thermo-responsive layer, as described herein. As noted, application of magnetic radiation to a thermo-responsive layer including magnetic nanoparticles may cause the magnetic nanoparticles to undergo magnetic hyperthermia thereby causing the magnetic nanoparticles and the thermo-responsive layer to increase in temperature relative to the temperature in a patient's body. The application of the magnetic radiation can continue at least until the magnetic nanoparticles and the thermo-responsive layer reach an elevated temperature at which the thermo-responsive layer is configured to liquify and mechanically detach from an adjacent material, as described herein.

As noted above, the selective liquification of the thermo-responsive layer may cause internal material, such as, but not limited to, sludge, bile, stone debris, etc., coupled to a surface (e.g., an inner surface) of the thermo-responsive layer (prior to application of the magnetic radiation) to be dislodge or slough off from the stent, thus mitigating occlusion of a lumen of the stent. It is contemplated that if sludge, bile, stone debris, biofilm, etc., are left undisturbed they may cause early occlusion or other malfunctioning of the stent 10.

It is contemplated that the elongated tubular member of the stent can be made from a number of different materials such as, but not limited to, metals, metal alloys, shape memory alloys, and/or polymers, as desired, enabling the stent to be expanded into shape when accurately positioned within the body. In some instances, the material may be selected to enable the stent to be removed with relative ease as well. For example, the elongated tubular member of the stent can be formed from alloys such as, but not limited to, nitinol and Elgiloy®. Depending on the material selected for construction, the stent may be self-expanding or require an external force to expand the stent. In some embodiments, composite filaments may be used to make the stent, which may include, for example, an outer shell or cladding made of nitinol and a core formed of platinum or other radiopaque material. It is further contemplated the elongated tubular member of the stent may be formed from polymers including, but not limited to, polyethylene terephthalate (PET). In some instances, the filaments of the stent, or portions thereof, may be bioabsorbable or biodegradable, while in other instances the filaments of the stent, or portions thereof, may be biostable and/or bio-inert.

The stents, delivery systems, and the various components thereof, may be made from a metal, metal alloy, polymer (some examples of which are disclosed below), a metal-polymer composite, ceramics, proteins, or combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material. Collagen is an example of a suitable protein.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), polycaprolactone (PCL), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent, the stent comprising:
an elongated tubular member forming a tubular wall, the elongated tubular member configured to move between a radially collapsed configuration and a radially expanded configuration;
a polymer coating disposed on a surface of the tubular wall;
a thermo-responsive layer disposed on an inner surface of the polymer coating; and
magnetic nanoparticles disposed on or within the thermo-responsive layer.

2. The stent of claim 1, wherein the polymer coating comprises silicone or polyurethane.

3. The stent of claim 1, wherein the polymer coating extends from a proximal end to a distal end of the elongated tubular member, and wherein the thermo-responsive layer extends from the proximal end to the distal end of the elongated tubular member.

4. The stent of claim 1, wherein the thermo-responsive layer comprises a thermo-responsive polymer or a thermo-responsive hydrogel.

5. The stent of claim 1, wherein the thermo-responsive layer comprises a collagen and polycaprolactone matrix.

6. The stent of claim 1, wherein the magnetic nanoparticles are uniformly dispersed on or embedded within the thermo-responsive layer along a length of the thermo-responsive layer.

7. The stent of claim 1, wherein the magnetic nanoparticles are arranged with a higher concentration of magnetic nanoparticles along a first region of the elongated tubular member than a concentration of the magnetic nanoparticles along a second region of the elongated tubular member.

8. The stent of claim 7, wherein the first region is adjacent to one or more structures forming the tubular wall, and wherein the second region is less proximate to the one or more structures forming the tubular wall.

9. The stent of claim 7, wherein the magnetic nanoparticles are dispersed on or are embedded within the thermo-responsive layer in a gradient extending from the first region to the second region.

10. The stent of claim 9, wherein the first region is located adjacent to a first end of the elongated tubular member and the second region is located adjacent to a second end of the elongated tubular member.

11. The stent of claim 1, wherein the magnetic nanoparticles have a number-average particle diameter in a range from about 1 nanometer to about 400 nanometers.

12. The stent of claim 1, wherein the magnetic nanoparticles comprise a silicone base and a magnetic material.

13. The stent of claim 1, wherein in response to an applied magnetic field, the magnetic nanoparticles are configured to be excited to an elevated temperature in a range from 42 degrees Celsius to 58 degrees Celsius, wherein the thermo-responsive layer is configured with a critical solution temperature or a melting point in the range from 42 degrees Celsius to 58 degrees Celsius, and wherein the applied magnetic field is a pulsed magnetic field or an alternating magnetic field.

14. The stent of claim 1, wherein the thermo-responsive layer comprises a plurality of thermo-responsive layers, the plurality of thermo-responsive layers including:
a second thermo-responsive layer disposed on an inner surface of the polymer coating, the second thermo-responsive layer configured to be excited to a second elevated temperature in response to an applied magnetic field; and
a first thermo-responsive layer disposed on an inner surface of the second thermo-responsive layer, the first thermo-responsive layer configured to be excited to a first elevated temperature in response to an applied magnetic field.

15. A stent, the stent comprising:
an elongated tubular member forming a tubular wall, the elongated tubular member configured to move between a radially collapsed configuration and a radially expanded configuration;
a polymer coating disposed on a surface of the tubular wall;
a thermo-responsive layer disposed on an inner surface of the polymer coating; and
magnetic nanoparticles disposed on or within the thermo-responsive layer,
wherein in response to one or more of the magnetic nanoparticles being excited to an elevated temperature by application of a magnetic field, at least a portion of the thermo-responsive layer is configured to liquefy and mechanically decouple from a remaining portion of the thermo-responsive layer, the polymer coating, or both.

16. A method for restoring patency of a stent implanted in a body of a patient, the method comprising:
applying a magnetic field to excite one or more magnetic nanoparticles in a thermo-responsive layer of the implanted stent to an elevated temperature, wherein the magnetic field is a pulsed magnetic field or an alternating magnetic field; and
wherein in response to the one or more magnetic nanoparticles being excited to the elevated temperature, at least a portion of the thermo-responsive layer is liquified.

17. The method of claim 16, wherein the liquified portion of the thermo-responsive layer is configured to mechanically decouple from a remaining portion of the thermo-responsive layer, a polymer coating of the implanted stent, or both.

18. The method of claim 16, wherein the one or more magnetic nanoparticles are configured to be exited to an elevated temperature in a range from 42 degrees Celsius to 58 degrees Celsius, and wherein the thermo-responsive layer is configured with a critical solution temperature or melting point in the range from 42 degrees Celsius to 58 degrees Celsius.

19. The method of claim 16, wherein the portion of the thermo-responsive layer that is configured to be liquified is further configured to remain liquified subsequent to cessation of applying the magnetic field.

20. The method of claim 16, wherein the magnetic field is applied exterior of the patient's body.
